(19) United States
(12) Patent Application Publication
(10) Pub. No.: US 2025/0093325 A1
(43) Pub. Date: Mar. 20, 2025

(54) DEVICES AND METHODS FOR MEASURING EROSION RESISTANCE OF ELECTROFUSED HIGH ZIRCONIA BRICKS

(71) Applicant: CAIHONG DISPLAY DEVICES CO., LTD., Xianyang (CN)

(72) Inventors: Longjiang ZHAO, Xianyang (CN); Wei YANG, Xianyang (CN); Jian XU, Xianyang (CN); Dacheng WANG, Xianyang (CN); Feng ZHANG, Xianyang (CN)

(73) Assignee: CAIHONG DISPLAY DEVICES CO., LTD., Xianyang (CN)

(21) Appl. No.: 18/967,579

(22) Filed: Dec. 3, 2024

Related U.S. Application Data
(63) Continuation-in-part of application No. PCT/CN2024/092914, filed on May 13, 2024.

(30) Foreign Application Priority Data
Sep. 4, 2023 (CN) 202311133606.8

(51) Int. Cl. G01N 33/38; C04B 35/48; C04B 35/484

(52) U.S. Cl. CPC G01N 33/38; C04B 35/481; C04B 35/484; C04B 2235/3244; C04B 2235/3418; C04B 2235/9669

(57) ABSTRACT

Devices and methods for measuring erosion resistance of electrofused high zirconia bricks are provided. The devices include a crucible, a crucible cover, a positioning axis, a plurality of holes, and a plurality of fixing members. The crucible cover is installed on an opening of the crucible, the positioning axis is installed at a center of the crucible cover, the plurality of holes are uniformly disposed along a circumferential direction of the crucible cover, and the plurality of fixing members are installed on the plurality of holes.

### CROSS-REFERENCE TO RELATED APPLICATION

This application is a continuation-in-part of International Application No: PCT/CN2024/092914, filed on May 13, 2024, which claims priority of Chinese Patent Application No. 202311133606.8 filed on Sep. 4, 2023, the contents of each of which are entirely incorporated herein by reference.

### TECHNICAL FIELD

The present disclosure relates to the field of refractory material performance testing, and in particular relates to devices and methods for measuring erosion resistance of electrofused high zirconia bricks.

### BACKGROUND

Glass products always have a wide range of applications in the display field. Without the support of the glass industry, the development of the display device industry will be limited. Although now there are other materials used on some occasions to replace the glass material, the excellent performance of the glass is not replaced. From the traditional color picture tube industry to the current panel display industry, glass is taken as a key component in the display device. In fact, the glass is not only the entire display device framework and carrier, but also optical components. As upper and lower substrates of the panel display device, the glass requires a fine micro-semiconductor technology processing process. To satisfy a high-efficiency and long-life operation of a furnace of high-generation, large-feed substrate glass, electrofused high zirconia bricks of the furnace must have a strong resistance to glass liquid erosion to satisfy the long-life operation of the furnace.

In the manufacturing process of the substrate glass, the glass compound is put into a feeding port of the furnace through a feeding system in a stable and smooth manner. Then the glass compound is melted, clarified, and homogenized in the furnace to provide the qualified and homogeneous glass liquid for a next process. The glass liquid melted in the furnace is alkali-free high-alumina borosilicate glass, which is mainly used as the substrate glass for panel display.

The electrofused high zirconia bricks, as a mainstream application material for a pool wall and a pool bottom of a gas-electric mixing furnace for substrate glass, with an increase in an amount of output of a high generation substrate glass, an increase in flow rate intensifies the scouring effect on the pool wall and bottom. Therefore, in the process of glass melting, the electrofused high zirconia bricks of the furnace inevitably suffer from erosions by the high temperature glass liquid and various electrochemical atmospheres in the furnace, resulting in a reduction in the life of the furnace. Therefore, research on the anti-glass erosion performance of the electrofused high zirconia bricks of the furnace has a very important application value in extending the life of the furnace and material selection and process formulation of the electrofused high zirconium bricks.

It is therefore necessary to provide devices and methods for measuring erosion resistance of the electrofused high zirconia bricks.

### SUMMARY

One or more embodiments of the present disclosure provide a device for measuring an erosion resistance of an electrofused high zirconia brick. The device includes a crucible, a crucible cover, a positioning axis, a plurality of holes, and a plurality of fixing members. The crucible cover is installed on an opening of the crucible, the positioning axis is installed at a center of the crucible cover, the plurality of holes are uniformly disposed along a circumferential direction of the crucible cover, and the plurality of fixing members are installed on the plurality of holes.

One or more embodiments of the present disclosure provide a method for measuring the erosion resistance of the electrofused high zirconia brick based on the device in the embodiments of the present disclosure. The method includes the following operations: processing an initial size of a sample to be measured of the electrofused high zirconia brick to obtain a processed sample; after adding a glass compound to the crucible, installing the crucible cover at an opening of the crucible, then inserting the processed sample into the glass compound through the holes and adopting the fixing member to fix the processed sample; placing the device as a whole in a high temperature furnace and insulating at a preset temperature, and measuring a size and depth change of the processed sample; and determining a glass erosion resistance rate of the processed sample based on the size and depth change.

### BRIEF DESCRIPTION OF THE DRAWINGS

The present disclosure will be further illustrated by way of exemplary embodiments, which will be described in detail with reference to the accompanying drawings. These embodiments are not limiting, and in these embodiments, the same counting denotes the same structure, wherein:

FIG. 1 is a schematic diagram illustrating a structure of a device for measuring an erosion resistance of an electrofused high zirconia brick according to some embodiments of the present disclosure;

FIG. 2 is a top view of a device for measuring an erosion resistance of an electrofused high zirconia brick according to some embodiments of the present disclosure;

FIG. 3 is a schematic diagram illustrating a structure of a device for measuring an erosion resistance of an electrofused high zirconia brick according to some embodiments of the present disclosure;

FIG. 4 is a schematic diagram illustrating a structure of a device for measuring an erosion resistance of an electrofused high zirconia brick according to some embodiments of the present disclosure; and

FIG. 5 is a schematic diagram illustrating an electrofused high zirconia brick before and after erosion according to some embodiments of the present disclosure.

### DETAILED DESCRIPTION

To more clearly illustrate the technical solutions of the embodiments of the present disclosure, the accompanying drawings required to be used in the description of the embodiments are briefly described below. Obviously, the accompanying drawings in the following description are only some examples or embodiments of the present disclosure, and it is possible for those skilled in the art to apply the present disclosure to other similar scenarios in accordance with these drawings without creative labor. Unless obviously obtained from the context or the context illustrates otherwise, the same numeral in the drawings refers to the same structure or operation.

It should be understood that the terms “system,” “device,” “unit,” and/or “module” as used herein are a way to distinguish between different components, elements, parts, sections, or assemblies at different levels. However, the words are replaced by other expressions if other words accomplish the same purpose.

As shown in the present disclosure and the claims, unless the context clearly suggests an exception, the words “a,” “one,” “an” and/or “the” are not specifically singular, but also include the plural. Generally, the terms “including” and “comprising” suggest only the inclusion of clearly identified operations and elements. In general, the terms “including” and “comprising” only suggest the inclusion of explicitly identified operations and elements that do not constitute an exclusive list, and the method or device also includes other operations or elements.

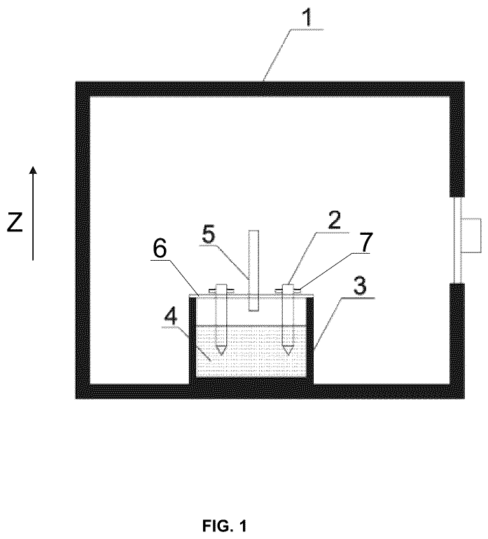

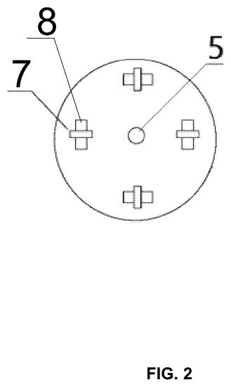

FIG. 1 is a schematic diagram illustrating a structure of a device for measuring an erosion resistance of an electrofused high zirconia brick according to some embodiments of the present disclosure. FIG. 2 is a top view of a device for measuring an erosion resistance of an electrofused high zirconia brick according to some embodiments of the present disclosure.

As shown in FIG. 1 and FIG. 2, the device for measuring an erosion resistance of an electrofused high zirconia brick includes a crucible 3, a crucible cover 6, a positioning axis 5, a plurality of holes 8, and a fixing member 7. In some embodiments, the crucible cover 6 is installed on an opening of the crucible 3, the positioning axis 5 is installed at a center of the crucible cover 6, the plurality of holes 8 are uniformly disposed along a circumferential direction of the crucible cover 6, and the fixing member 7 is installed on the plurality of holes 8.

The crucible 3 is used as a container to accommodate other materials. For example, the crucible 3 is used for accommodating a glass compound 4 that erodes an electrofused high zirconia brick sample 2. In some embodiments, an accommodation space is formed inside the crucible 3. In some embodiments, the accommodation space has an opening disposed in a vertical direction facing upward (e.g., a Z direction in FIG. 1). The opening is used to add other substances to the crucible 3. In some embodiments, the crucible 3 has a plurality of shapes, for example, at least one of a cylinder, a prism, etc.

In some embodiments, the crucible 3 is made of various materials, for example, at least one of a metal, a ceramic, etc.

Merely by way of example, the crucible 3 adopts a platinum-rhodium alloy (PtRh20), which ensures a strength and chemical stability of the crucible 3 at a high temperature and avoids the crucible 3 from corrosion in a high temperature environment and affecting the experimental accuracy.

In some embodiments, the crucible 3 is fitted with a matching crucible cover 6 at the opening of the crucible 3.

The crucible cover 6 is used to close or open the opening of the crucible 3. In some embodiments, the crucible cover 6 is shaped to fit the opening of the crucible 3, for example, rectangular, circular, etc. In some embodiments, the crucible cover 6 is a flat plate-shaped structure.

In some embodiments, the crucible cover 6 is capable of serving as a mounting base for mounting other objects or structures, for example, mounting the electrofused high zirconia brick sample 2. In some embodiments, at least a portion of the electrofused high zirconia brick sample 2 passes through the crucible cover 6 and extends into an interior of the crucible 3.

In some embodiments, the crucible 3 is disposed within the high temperature furnace 1, and the interior of the high temperature furnace 1 forms the high temperature environment for heating the crucible 3 and the material within the crucible 3, or for keeping the crucible 3 and the material within the crucible 3 at a certain temperature.

The positioning axis 5 is used as a reference standard to facilitate a positioning of the plurality of holes 8, thereby determining shapes and positions of the holes 8 when processing the holes 8, which is conducive to improving the setting accuracy of the holes 8. In some embodiments, the positioning axis 5 has a plurality of shapes, for example, at least one of prismatic, cylindrical, etc. In some embodiments, an axial direction of the positioning axis 5 is disposed perpendicular to the crucible cover 6.

A hole 8 refers to a through hole disposed on the crucible cover 6, which is used for the electrofused high zirconia brick sample 2 to pass through.

In some embodiments, the hole 8 is square and slightly greater than, in size, the electrofused high zirconia brick sample 2 so as to be easily adapted to fit the shape of the electrofused high zirconia brick sample 2 such that the electrofused high zirconia brick sample 2 is able to be inserted in the hole 8.

In some embodiments, sizes of the crucible 3, as well as the holes 8, are matched to the size of the electrofused high zirconia brick sample 2 to satisfy the measurement requirement.

In some embodiments, a length of a hole 8 is greater than 105 mm and a width of the hole 8 is greater than 24 mm.

In some embodiments, a depth of the crucible 3 is greater than 12 mm.

Exemplarily, the size of the electrofused high zirconia brick sample 2 is 105 mm×24 mm×12 mm, the length of the hole 8 is 106 mm, the width of the hole 8 is 25 mm, and the depth of the crucible 3 is 13 mm so as to be able to match the size of the electrofused high zirconia brick sample 2.

In some embodiments, the holes 8 are disposed uniformly around the positioning axis 5 along a circumference of the crucible cover 6. In some embodiments, there are 4 holes 8. In this manner, four electrofused high zirconia brick samples 2 are mounted on the crucible cover 6 at the same time for experimentation. In some embodiments, there are other counts of holes 8, for example, 6, 8, etc. The count of the holes 8 is set according to actual requirements. In some embodiments, as shown in FIG. 2, when the holes 8 are square, an angle between a length direction of the holes 8 and a radial direction of the crucible cover 6 is 90 degrees.

In some embodiments, a hole 8 is disposed with a fixing member 7. The fixing member 7 is used to fix the electrofused high zirconia brick sample 2. In some embodiments, the fixing member 7 is detachable connected to the electrofused high zirconia brick sample 2 in various manners, for example, at least one of a snap-fit, a buckle-fit, etc.

Merely by way of example, the fixing member 7 is a rod-like structure. The fixing member 7 penetrates the electrofused high zirconia brick sample 2 along the radial direction of the crucible cover 6. The fixing member 7 is unable to pass through the hole 8. The fixing member 7 is snapped to an upper surface of the crucible cover 6 when the electrofused high zirconia brick sample 2 passes through the hole 8.

In some embodiments, by utilizing existing laboratory parts and their size layouts, the device for measuring the erosion resistance of an electrofused high zirconia brick is rationally designed, so as to enable a simple structure and an easy operation of the measurement device.

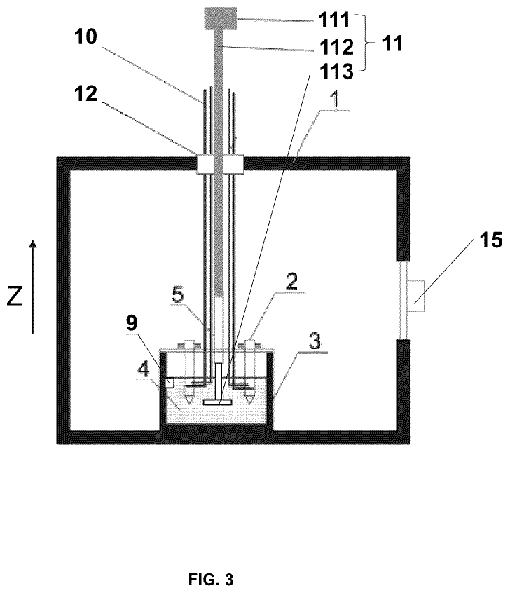

FIG. 3 is a schematic diagram illustrating a structure of a device for measuring an erosion resistance of an electrofused high zirconia brick according to some embodiments of the present disclosure.

In some embodiments, as shown in FIG. 3, the device for measuring the erosion resistance of the electrofused high zirconia brick further includes a temperature sensor 9, a measuring device 10, and a stirring device 11.

The temperature sensor 9 is used to obtain temperature data, for example, the temperature data of the glass compound 4. In some embodiments, the temperature sensor 9 is disposed on the crucible 3. In some embodiments, there is a plurality of temperature sensors 9, and the plurality of temperature sensors 9 are disposed at different positions on the crucible 3.

The measuring device 10 is used to measure an electrofused high zirconia brick sample 2 to obtain erosion data.

The erosion data refers to data related to the electrofused high zirconia brick sample 2 after the electrofused high zirconia brick sample 2 is eroded. In some embodiments, the erosion data includes at least one of a thickness, a surface area, a surface roughness, etc. of the electrofused high zirconia brick sample 2 after being eroded.

In some embodiments, the measuring device 10 includes a plurality of probes and/or probe rods arranged in an array, and the probes and/or probe rods are inserted in the holes 8. In some embodiments, the probes and/or probe rods are inserted into the crucible 3 through a window 12 opened on the high temperature furnace 1. The window 12 is used as a mounting base for mounting the measuring device 10 and the stirring device 11. In some embodiments, the probes and/or the probe rods are inserted into the crucible 3 through through-holes separately opened on the crucible cover 6.

The probes and/or probe rods refer to structures used to obtain the erosion data. For example, the probes and/or probe rods are test probes, etc.

In some embodiments, the measuring device 10 moves in a length direction (e.g., the Z direction in FIG. 3) of the positioning axis 5. In some embodiments, the movement of the measuring device 10 is driven based on various manners. For example, the measuring device 10 is manually driven, driven by mechanical structure, etc. The mechanical structure includes at least one of a hydraulic cylinder, an air cylinder, an electric push cylinder, etc.

In some embodiments, the measuring device 10 is connected to the stirring device 11 and rotates together with a rotation of the stirring device 11.

In some embodiments, the measuring device 10 is disposed as retractable, with an end near the crucible 3 being able to extend and insert into the glass compound 4, or the end is able to be retracted and does not contact the glass compound 4. The extension and retraction of the measuring device 10 are manually driven or driven by a driving mechanism (e.g. a motor).

The stirring device 11 is used to stir the glass compound 4 in the crucible 3 to make the glass compound 4 flow, thereby creating a scenario in which the electrofused high zirconia brick sample 2 is dynamically eroded. In some embodiments, at least a portion of the stirring device 11 extends through the crucible cover 6 and into an interior of the crucible 3.

In some embodiments, the stirring device 11 is configured to operate based on a stirring parameter. The stirring parameter refers to a parameter associated with the stirring device 11. In some embodiments, the stirring parameter includes at least one of a stirring speed, a stirring time, a stirring frequency, etc. In some embodiments, the stirring parameter is a preset value, which is set according to actual requirements.

In some embodiments, the stirring device 11 includes a stirring motor 111, a stirring rod 112, and a stirring paddle 113. The stirring motor 111 is located outside the high temperature furnace, and an output axis of the stirring motor 111 is connected to one end of the stirring rod 112. The other end of the stirring rod 112 is connected to the stirring paddle 113 and inserted into the glass compound 4.

In some embodiments, the stirring rod 112 is fixedly connected to the positioning axis 5. In some embodiments, the stirring rod 112 is integrated into the positioning axis 5. In some embodiments, a through hole is disposed within the positioning axis 5, and at least a portion of the stirring device 9 passes through the through hole in the positioning axis 5 and extends into the interior of the crucible 3.

Using the temperature sensor facilitates real-time monitoring of the temperature inside the crucible and enables a timely response when there are abnormal changes in the temperature inside the crucible. By using the measurement device to obtain the erosion data in real time in the process of the experiment, an interruption of the experiment when the experimental data needs to be obtained is avoided, which is conducive to an improvement of experimental efficiency. By using the stirring device to stir the glass compound in the crucible, so as to simulate the dynamic erosion, a scope of application of the device for measuring the erosion resistance of electrofused high zirconia brick is improved.

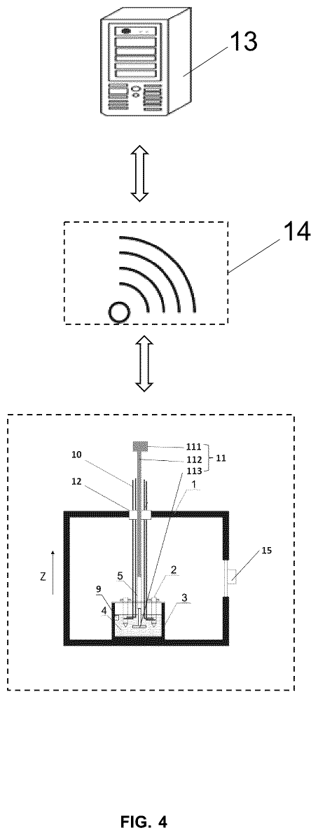

FIG. 4 is a schematic diagram illustrating a structure of a device for measuring an erosion resistance of an electrofused high zirconia brick according to some embodiments of the present disclosure.

In some embodiments, as shown in FIG. 4, the device for measuring the erosion resistance of the electrofused high zirconia brick further includes a remote processor 13, a communication device 14, and the high temperature furnace 1.

The remote processor 13 is used to process data, generate a control instruction and issue the control instruction to an actuator to control the actuator to perform a corresponding action. In some embodiments, the actuator includes the communication device 14, the high temperature furnace 1, the stirring device 11, the temperature sensor 9, and the measuring device 10.

In some embodiments, the communication device 14 is configured to transmit data between the stirring device 11, the temperature sensor 9, the measuring device 10, and the remote processor 13. The remote processor 13 is capable of controlling the temperature sensor 9, the measuring device 10, and the stirring device 11, respectively.

In some embodiments, the remote processor 13 is configured to determine an erosion experimental parameter.

The erosion experimental parameter refers to a parameter associated with the erosion experiment. In some embodiments, the erosion experiment parameter includes a temperature parameter. In some embodiments, the temperature parameter includes a warming rate, a temperature holding time, etc. In some embodiments, the temperature parameter is a preset value, which is set according to actual requirements.

In some embodiments, the high temperature furnace 1 is configured to perform a temperature regulation on the glass compound 4 based on the temperature parameter.

By reasonably determining the erosion experimental parameter, the success rate and the effectiveness of the experiment are improved, and the consumption of the experimental device is reduced, thereby reducing the experimental time.

In some embodiments, the erosion experimental parameter further includes a stirring parameter. More contents on the stirring parameter may be found in the previous related descriptions.

In some embodiments, the remote processor 13 is further configured to determine the erosion experimental parameter based on data of the glass compound and data of the electrofused high zirconia brick sample.

The data of the glass compound refers to data related to the glass compound 4, for example, a composition, a ratio, etc., of the glass compound 4. The data of the glass compound is obtained by performing a composition test on the glass compound and manually entered to the remote processor 13.

The data of the electrofused high zirconia brick sample (hereinafter referred to as “sample data”) refers to data relating to the electrofused high zirconia brick sample 2. For example, the composition, the ratio, etc., of the electrofused high zirconia brick sample 2. The sample data is obtained by performing the composition test on the electrofused high zirconia brick sample 2 and manually entered into the remote processor 13.

In some embodiments, the remote processor 13 determines the erosion experimental parameter in various manners. For example, the remote processor 13 constructs a sample vector based on the data of the glass compound and the sample data. The remote processor 13 constructs standard vectors based on historical data. A standard vector includes data of historical glass compound and historical sample data, and the remote processor 13 constructs a vector database based on the standard vectors as well as historical stirring parameters corresponding to the standard vectors. The remote processor matches, based on the sample vector, a standard vector with the highest similarity degree to the sample vector from the vector database, and determines the historical stirring parameter corresponding to the standard vector as the stirring parameter of the sample vector.

As another example, the remote processor 13 controls the stirring speed of the stirring device 11, so that the stirring speed gradually increases until the stirring speed complies with the production requirements of an actual production line. The remote processor 13 confirms the stirring speed as the stirring parameter.

Based on the data of the glass compound and the sample data, the historical data is utilized for determining the erosion experimental parameter, which is conducive to improving the efficiency and accuracy of determining the erosion experimental parameter. By determining the stirring parameter, the stirring device is used to stir the glass compound to simulate a real production scenario, thus making the experiment more comply with the production scenario.

In some embodiments, the remote processor 13 is further configured to determine a sample size of the electrofused high zirconia brick sample 2 and determine erosion experimental parameter based on the sample size, the data of the glass compound, and the sample data.

The sample size refers to a parameter related to a size of the electrofused high zirconia brick sample 2. For example, a thickness of the electrofused high zirconia brick sample 2, etc. In some embodiments, the sample size is measured in real time by the measuring device 10. More contents on the measuring device 10 may be found in the previous description.

In some embodiments, the remote processor 13 determines the erosion experimental parameter in various manners. For example, the erosion experimental parameter is determined according to the actual requirements based on, on the production line, the actual sample size of the electrofused high zirconia brick sample 2, the actual data of the glass compound, and the actual sample data of the electrofused high zirconia brick sample 2.

Based on, on the production line, the actual sample size of the electrofused high zirconia brick sample 2, the actual data of the glass compound, and the actual sample data of the electrofused high zirconia brick sample 2, the erosion experimental parameter is determined so that the erosion experimental parameter are more in line with the simulated production scenario.

In some embodiments, the remote processor 13 is further configured to determine an equivalent erosion experimental parameter based on current data of the glass compound and current sample data of the electrofused high zirconia brick sample 2.

The equivalent erosion experimental parameter refers to a relevant parameter for performing an equivalent experiment. The equivalent erosion experimental parameter includes a temperature parameter, a stirring parameter, etc. in the equivalent experiment.

In some embodiments, the remote processor 13 determines the equivalent erosion experimental parameter in various manners. For example, the remote processor 13 takes the experiment in the historical data in which historical data of the glass compound and historical sample data are the same as the current data of the glass compound and the current sample data as a previous sample. The remote processor 13 may determine, based on the previous sample, a fitting curve with time as an independent variable, and the historical erosion experimental parameters as a dependent variable of the fitting curve. By querying the fitting curve, the remote processor 13 determines a corresponding historical erosion experiment parameter based on a current required experiment time and determines the corresponding historical erosion experiment parameter as the equivalent erosion experimental parameter.

By determining the equivalent erosion experimental parameter, the equivalent experiment is performed to shorten the duration of the experiment and improve the efficiency.

In some embodiments, the remote processor 13 is further configured to determine, by a determination model, an erosion degree, based on the current data of the glass compound, the current sample data, candidate equivalent erosion experimental parameters, and an expected experimental duration; and determine, based on the erosion degree, the equivalent erosion experimental parameter. The determination model is a machine learning model.

The candidate equivalent erosion experimental parameters refer to a plurality of equivalent erosion experimental parameters to be selected. In some embodiments, the candidate equivalent erosion experimental parameters include equivalent erosion experimental parameters determined based on the fitted curve, erosion experimental parameters corresponding to erosion experiments whose duration meets the requirements in the historical data, and erosion experimental parameters generated by a random variation of the standard erosion experimental parameters.

The expected experimental duration refers to an expected duration of the experiment. In some embodiments, the expected experimental duration is a preset value, which is preset according to actual requirements.

The erosion degree refers to correlation data used to characterize a degree to which the electrofused high zirconia brick sample 2 is eroded. In some embodiments, the erosion degree is percentage data.

In some embodiments, the remote processor 13 determines the erosion degree in various manners. For example, the remote processor 13 determines the erosion degree based on a determination model.

The determination model refers to a model used to determine the erosion degree. In some embodiments, the determining model is a machine learning model.

In some embodiments, input to the determination model includes the current data of the glass compound, the current sample data, the candidate equivalent erosion experimental parameters, the expected experimental duration, and output of the determination model includes the erosion degree.

In some embodiments, the remote processor 13 trains the determination model based on a first training dataset by, for example, a gradient descent manner.

In some embodiments, the first training dataset includes first training samples and a first training label corresponding to each first training sample.

In some embodiments, a first training sample includes historical data of the glass compound, historical sample data, a historical equivalent erosion experimental parameter, and a historical experimental duration in the historical data. In some embodiments, a first training label is as follows:

$$B = 1 - \frac{{H}_{2}}{{H}_{1}} \qquad ( 1 )$$

B denotes the first training label, H₁ denotes an initial thickness of a corresponding electrofused high zirconia brick sample 2 under a first training sample condition, and H₂ denotes a thickness of the electrofused high zirconia brick 2 after experiment under the first training sample condition.

In some embodiments, the determination model is trained in the following manner: a first training sample with a first training label is input to an initial determination model; a loss function is constructed based on the first training label and a prediction result of the initial determination model; the initial determination model is iteratively updated based on the loss function, and training of the determination model is completed when the loss function satisfies a preset condition. The preset condition is that the loss function converges, a count of iterations reaches a set value, etc.

In some embodiments, the remote processor 13 determines the equivalent erosion experiment parameter based on the erosion degree. For example, the remote processor 13 determines an erosion degree that satisfies a preset condition and determines the candidate equivalent erosion experimental parameter corresponding to the erosion degree as the equivalent erosion experimental parameter. The preset condition includes that the erosion degree is greater than an erosion degree threshold. In some embodiments, the erosion degree threshold is a preset value that is set according to actual requirements.

Using the determination model improves the accuracy of determining the erosion degree, thus improving the accuracy of the subsequent determination of the equivalent erosion experiment parameter.

Using the remote processor enables an automated control, thereby improving the automation of the device for measuring the erosion resistance of the electrofused high zirconia brick, as well as the efficiency of the experiment. By pre-determining the erosion experimental parameter, the experimental parameter is scientifically and efficiently set to improve the success rate of the experiment and reduce the waste of experimental equipment and a waste of time.

In some embodiments, the device for measuring the erosion resistance of the electrofused high zirconia brick further includes a speed detection device 15. In some embodiments, the speed detection device 15 is disposed outside the high temperature furnace 1 and is configured to obtain a glass liquid speed. In some embodiments, the speed detection device 15 and the glass compound 4 are not in contact.

The speed detection device refers to a device used to measure a speed. For example, the speed detection device includes an optical Doppler velocimeter, an ultrasonic velocimeter, an eddy current sensor, etc.

Merely by way of example, when the speed detection device 15 adopts the optical Doppler velocimeter or the ultrasonic velocimeter, a velocimeter window is set on the high temperature furnace 1, and velocimetry signals issued by the optical Doppler velocimeter or the ultrasonic velocimeter are sent to the crucible 3 through the velocimeter window. The velocimetry signals are reflected by the glass compound 4 in the crucible 3 and then received by the optical Doppler velocimeter or the ultrasonic velocimeter through the velocimeter window, so that the optical Doppler velocimeter or the ultrasonic velocimeter is able to detect the glass liquid speed of the glass compound 4.

In some embodiments, the remote processor 13 is further configured to determine, based on the erosion experimental parameter, a first control parameter and initiate the experiment. The remote processor 13 may be further configured to periodically obtain the glass liquid speed, a glass liquid temperature, and the erosion data; and determine a second control parameter based on the glass liquid speed, the glass liquid temperature and the erosion data. The glass liquid temperature is obtained by the temperature sensor 9. The erosion data is obtained by the measuring device 10. More contents about the erosion data may be found in the previous related descriptions.

The first control parameter refers to a control instruction that includes the erosion experiment parameter. The remote processor 13 controls, based on the first control parameter, the high temperature furnace 1 to heat the glass compound 4 with a corresponding temperature parameter in the erosion experimental parameter and/or control the stirring device 11 to stir the glass compound 4 with the corresponding stirring parameter in the erosion experimental parameter.

In some embodiments, the first control parameter is a preset value. The remote processor 13 determines the first control parameter in various manners. For example, the first control parameter is determined by at least one of obtaining a manual input, obtaining from the historical data, etc.

The second control parameter refers to a control instruction after the erosion experiment parameter is updated. The remote processor 13 controls, based on the second control parameter, the high temperature furnace 1 to regulate the temperature based on the updated erosion experimental parameter and/or control the stirring device 11 to regulate the stirring speed of the glass compound 4 based on the updated erosion experimental parameter.

In some embodiments, the remote processor 13 determines the second control parameter in various manners. For example, the updated erosion experimental parameter is determined based on the glass liquid temperature of the glass compound, the glass liquid speed of the glass compound, and the erosion data, and the updated erosion experimental parameters are converted into a machine instruction as the second control parameter.

In some embodiments, the remote processor determines the second control parameter in other means. More contents may be referred to in the related descriptions below.

In some embodiments, a length of the cycle in which the remote processor 13 periodically obtains the glass liquid speed, the glass liquid temperature, and the erosion data is related to the glass liquid speed and the glass liquid temperature of a plurality of cycles before the current cycle.

In some embodiments, when a variance of the glass liquid speeds and/or a variance of the glass liquid temperatures of the plurality of cycles is less than a first threshold, the remote processor 13 elongates the length of the cycle.

In some embodiments, when the variance of the glass liquid speeds and/or the variance of the glass liquid temperatures of the plurality of periods is less than a second threshold, the remote processor 13 shortens the length of the cycle.

In some embodiments, a magnitude by which the remote processor 13 elongates or shortens the length of the cycle is set according to the actual requirements. In some embodiments, the first threshold and the second threshold are preset values that are set according to the actual needs. In some embodiments, the first threshold is less than the second threshold.

By adjusting the length of the cycle, the whole experimental process is regulated more accurately and efficiently, and the effectiveness of the experiment is improved.

By using the speed detection device, the speed of the glass compound is detected in real time to avoid the speed of the glass compound being too fast to cause bubbles and affect the experiment's accuracy, or avoid the speed of the glass compound being too slow, which makes it impossible to simulate a real corrosion situation.

In some embodiments, the remote processor 13 is further configured to determine, through an updating model, the second control parameter based on the glass liquid speed, the glass liquid temperature, and the erosion data.

The updating model refers to a model for determining the second control parameter. In some embodiments, the updating model is a machine learning model.

In some embodiments, input to the updating model includes the glass liquid speed, the glass liquid temperature, and the erosion data, and output of the updating model includes the second control parameter.

In some embodiments, the input to the updating model further includes the data of the glass compound, the sample data, and air composition in the high temperature furnace.

The air composition in the high temperature furnace refers to data related to air inside the furnace. For example, the air composition includes percentages of nitrogen, oxygen, carbon dioxide, etc.

By taking the data of the glass compound and the sample data as the input of the updating model, when determining the second control parameter, the influence of the glass composition and the sample composition on the corrosion situation is considered. By taking the air composition in the furnace as the input of the updating model, oxidative corrosion caused by the oxygen in the air being incorporated into the glass liquid is considered, which improves the accuracy of the determined second control parameter.

In some embodiments, the remote processor 13 trains the updating model based on a second training dataset by, for example, the gradient descent manner.

In some embodiments, the second training dataset includes second training samples and a second training label corresponding to each second training sample.

In some embodiments, a second training sample included a historical glass liquid speed, a historical glass liquid temperature, and historical erosion data in the historical data, and the second training sample corresponds to a second control parameter for the current experimental cycle. A second training label corresponding to the second training sample is a second control parameter for a next experimental cycle corresponding to the second training sample.

In some embodiments, a training process of the updating model is similar to the training process of the determination model, which may refer to the previous related description of the training process of the determination model.

Using the updating model to determine the second control parameter improves the accuracy and efficiency of determining the second control parameter.

In some embodiments, the remote processor 13 is further configured to determine a measurement time of the measuring device 10, the measurement time being related to the data of the glass compound, the sample data, and the erosion experimental parameter.

The measurement time refers to a parameter related to time-related parameter when the measurement device measures the electrofused high zirconia brick sample 2. In some embodiments, the measurement time includes at least one of a measurement start time, a measurement end time, a measurement duration, a measurement interval, etc.

In some embodiments, the remote processor 13 obtains, in the historical data, a historical experiment matching the current data of the glass compound, the sample data, and the erosion experimental parameter, and plots an erosion curve based on the erosion data at each time point in the experimental data corresponding to the historical experiment. A horizontal coordinate of the erosion curve is the time point, and a vertical coordinate is the erosion data. If the erosion curve does not have an inflection point, the remote processor 13 controls the measurement device to take measurements according to a preset time interval. If there is an inflection point in the erosion curve, the remote processor 13 adjusts the measurement time according to the inflection point. For example, the remote processor determines a historical time point at which the inflection point occurs, and a preset measurement start time of the current experiment corresponding to the historical time point, if the preset measurement start time has not yet arrived, the remote processor advances the preset measurement start time, and the degree of advancement is set according to the actual requirements.

The matching refers to that the current data of the glass compound is consistent with the historical data of the glass compound, the sample data is consistent with the historical sample data, and the erosion experimental parameter is consistent with the historical erosion experimental parameter, or the similarity thereof is greater than a similarity threshold. In some embodiments, the similarity threshold is a preset value that is set according to the actual requirements. For example, 90%, etc.

By making the measurement time correlate with the data of the glass compound, the sample data, and the erosion experimental parameter, the measurement time may be regulated according to the actual data of glass compound, actual sample data, and actual erosion experimental parameter in combination with the historical data, so as to facilitate the regulation of the experiment process according to actual measurement data, which is conducive to improving the accuracy and effectiveness of the experiment.

Some embodiments of the present disclosure provide a method for measuring the erosion resistance of the electrofused high zirconia brick, the method including the following steps.

In step S1, an initial size of a sample to be measured of the electrofused high zirconia brick is processed to obtain a processed sample.

In some embodiments, the sample to be measured of the electrofused high zirconia brick is first processed to obtain the processed sample with a size of 105 mm×24 mm×12 mm.

In step S2, after the glass compound 4 is added to the crucible 3, the crucible cover 6 is installed in the opening of the crucible 3, and then the processed sample is inserted into the glass compound 4 through a hole 8, and the fixing member 7 is used to fix the processed sample. In some embodiments, a chemical component of the sample to be measured of the electrofused high zirconia brick includes ZrO₂, SiO₂, and impurities, with a mass percentage of ZrO₂ not less than 93%, a mass percentage of SiO₂ not less than 4%, and a mass percentage of the impurities greater than 0% and not greater than 3%, and a sum of the mass percentages of ZrO₂, SiO₂, and the impurities is 100%. In some embodiments, the glass compound includes a glass raw material and a crushed glass. In some embodiments, a mass fraction ratio of the glass raw material and the crushed glass is (75%-76%):(24%-25%).

In step S3, the device as a whole is placed in the high temperature furnace 1, and after being insulated at a preset temperature, a size and depth change of the processed sample is measured.

In some embodiments, the preset temperature is 1600-1700° C. In some embodiments, the preset temperature is 1630-1670° C. In some embodiments, the preset temperature is 1650° C.

In some embodiments, a duration of the insulation is 65-80 h. In some embodiments, the duration of the insulation is 68-78 h. In some embodiments, the duration of the insulation is 70-75 h. In some embodiments, the duration of the insulation is 72 h.

In step S4, a glass erosion resistance rate of the processed sample is determined based on the size and depth change.

In some embodiments, the chemical component of the sample to be measured of the electrofused high zirconia brick includes ZrO₂, SiO₂, and the impurities. The mass percentage of ZrO₂ is not less than 93%, the mass percentage of SiO₂ is not less than 4%, the mass percentage of the impurities is greater than 0% and not greater than 3%, and the sum of the mass percentages of ZrO₂, SiO₂ and the impurities is 100%.

In some embodiments, the glass component includes the glass raw material and the crushed glass.

In some embodiments, the mass fraction ratio of the glass raw material and the crushed glass is (75%-76%):(24%-25%). In some embodiments, the mass fraction ratio of the glass raw material and the crushed glass is (75.1%-75.9%):(24.1%-24.9%). In some embodiments, the mass fraction ratio of the glass raw material and the crushed glass is (75.2%-75.8%):(24.2%-24.8%). In some embodiments, the mass fraction ratio of the glass raw material and the crushed glass is (75.3%-75.7%):(24.3%-24.7%). In some embodiments, the mass fraction ratio of the glass raw material and the crushed glass is (75.4%-75.6%):(24.4%-24.6%). In some embodiments, the mass fraction ratio of the glass raw material and the crushed glass is 75.5%: 24.5%.

In some embodiments, in the method for measuring the erosion resistance of the electrofused high zirconia brick, by measuring the size and depth change of the electrofused high zirconia brick sample before and after the erosion, the glass erosion resistance rate of the electrofused high zirconia brick is determined. The result of the measurement is scientifically reliable, which has a very important application value in extending the service life of the furnace, the material selection and the process development of the electrofused high zirconia brick.

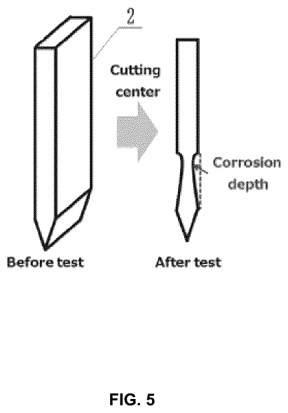

FIG. 5 is a schematic diagram illustrating an electrofused high zirconia brick before and after erosion according to some embodiments of the present disclosure. Exemplarily, experiments are conducted with two electrofused high zirconia brick samples, and the experimental data are shown in Table 1 below.

TABLE 1
Erosion rates of electrofused high zirconia brick samples

| Size changes before and after erosion | Sample 1 | Sample 2 |
|---|---|---|
| Size before erosion/mm | 11.88 mm | 11.96 mm |
| Size after erosion/mm | 9.12 mm | 9.38 mm |
| Erosion amount/mm | 2.76 mm | 2.58 mm |
| Erosion rate mm/12 h | 0.46 mm/12 h | 0.43 mm/12 h |

In this embodiment, the ratio of glass compound 4 in the experiment and the experimental temperature are kept being the same as the ratio and the experimental temperature of the production line. That is, a mass fraction ratio of the glass raw material and the crushed glass is about 75.5%:24.5%, and the device as a whole is placed in the high temperature furnace at a temperature of 1650° C. and insulated for 72 h. By weight, the sample 1 of the embodiment contains 84% of zirconia and 10% of glass phase, and the sample 2 of the embodiment contains 94% of zirconia and 5% of glass phase.

As shown in FIG. 5 and Table 1, by measuring the size and depth change of the electrofused high zirconia brick sample before and after erosion, the glass erosion resistance rate of the electrofused high zirconia brick is accurately determined.

The basic concepts have been described above, and it is apparent to those skilled in the art that the foregoing detailed disclosure is intended as an example only and does not constitute a limitation of the present disclosure. While not expressly stated herein, those skilled in the art may make various modifications, improvements, and amendments to the present disclosure. Those types of modifications, improvements, and amendments are suggested in the present disclosure, so those types of modifications, improvements, and amendments remain within the spirit and scope of the exemplary embodiments of the present disclosure.

Also, the present disclosure uses specific words to describe embodiments thereof. Such as “an embodiment,” “one embodiment,” and/or “some embodiments” means a feature, structure, or characteristic associated with at least one embodiment of the present disclosure. Accordingly, it should be emphasized and noted that two or more references in the present disclosure, at different positions, to “one embodiment” or “an embodiment” or “an alternative embodiment” in different places in the present disclosure do not necessarily refer to the same embodiment. In addition, certain features, structures, or characteristics in one or more embodiments of the present disclosure are suitably combined.

Finally, it should be understood that the embodiments described in the present disclosure are only used to illustrate the principles of the embodiments of the present disclosure. Other deformations also fall within the scope of the present disclosure. As such, alternative configurations of embodiments of the present disclosure are viewed as consistent with the teachings of the present disclosure as an example, not as a limitation. Correspondingly, the embodiments of the present disclosure are not limited to the embodiments expressly presented and described herein.

What is claimed is:

1. A device for measuring an erosion resistance of an electrofused high zirconia brick, comprising: a crucible, a crucible cover, a positioning axis, a plurality of holes, and a plurality of fixing members; wherein
the crucible cover is installed on an opening of the crucible, the positioning axis is installed at a center of the crucible cover, the plurality of holes are uniformly disposed along a circumferential direction of the crucible cover, and the plurality of fixing members are installed on the plurality of holes.

2. The device of claim 1, wherein the plurality of holes are square, a length of each of the plurality of holes is greater than 105 mm, and a width of each of the plurality of holes is greater than 24 mm.

3. The device of claim 1, wherein a depth of the crucible is greater than 12 mm, and a material of the crucible is platinum-rhodium alloy (PtRh20).

4. The device of claim 1, wherein the plurality of holes are square, and an angle between a length direction of each of the plurality of holes and a radial direction of the crucible cover is 90 degrees.

5. The device of claim 1, further comprising:
a temperature sensor disposed within the crucible;
a measuring device including a plurality of probes or probe rods arranged in an array, the plurality of probes or probe rods being inserted in the plurality of holes; and
a stirring device configured to operate based on a stirring parameter.

6. The device of claim 5, further comprising a remote processor, a communication device, and a high temperature furnace, wherein
the communication device is configured to perform a data transmission between the stirring device, the temperature sensor, the measuring device, and the remote processor,
the remote processor is configured to determine an erosion experimental parameter, the erosion experimental parameter including a temperature parameter, and
the high temperature furnace is configured to perform a temperature regulation on a glass compound based on the temperature parameter.

7. The device of claim 6, wherein
the erosion experimental parameter further includes the stirring parameter, and
the remote processor is further configured to determine the erosion experimental parameter based on data of the glass compound and sample data of an electrofused high zirconia brick sample.

8. The device of claim 7, wherein the remote processor is further configured to:
determine a sample size of the electrofused high zirconia brick sample; and
determine the erosion experimental parameter based on the sample size, the data of the glass compound, and the sample data.

9. The device of claim 7, wherein the remote processor is further configured to determine an equivalent erosion experimental parameter based on current data of the glass compound and current sample data.

10. The device of claim 7, further comprising a speed detection device disposed outside the high temperature furnace and is configured to obtain a glass liquid speed, wherein the remote processor is further configured to:
determine a first control parameter based on the erosion experimental parameter;
initiate an experiment;
periodically obtain the glass liquid speed, a glass liquid temperature, and erosion data; and
determine a second control parameter based on the glass liquid speed, the glass liquid temperature, and the erosion data.

11. The device of claim 10, wherein the remote processor is further configured to determine, through an updating model, the second control parameter based on the glass liquid speed, the glass liquid temperature, and the erosion data, the updating model being a machine learning model.

12. The device of claim 11, wherein an input of the updating model includes the data of the glass compound, the sample data, and an air composition within the high temperature furnace.

13. The device of claim 11, wherein a length of a cycle in which the remote processor periodically obtains the glass liquid speed, the glass liquid temperature, and the erosion data is related to glass liquid speeds and glass liquid temperatures of a plurality of cycles before a current cycle.

14. The device of claim 6, wherein the remote processor is further configured to determine a measurement time of the measuring device, the measurement time being related to data of the glass compound, sample data of an electrofused high zirconia brick sample, and the erosion experimental parameter.

15. A method for measuring an erosion resistance of an electrofused high zirconia brick based on a device for measuring the erosion resistance of the electrofused high zirconia brick, wherein
the device includes a crucible, a crucible cover, a positioning axis, a plurality of holes, and a plurality of fixing members, wherein
the crucible cover is installed on an opening of the crucible, the positioning axis is installed at a center of the crucible cover, the plurality of holes are uniformly disposed along a circumferential direction of the crucible cover, and the plurality of fixing members are installed on the plurality of holes,
the method includes:
processing an initial size of a sample to be measured of the electrofused high zirconia brick to obtain a processed sample;
after adding a glass compound to the crucible, installing the crucible cover at an opening of the crucible, then inserting the processed sample into the glass compound through one of the plurality of holes and adopting a fixing member corresponding to the hole to fix the processed sample;
placing the device as a whole in a high temperature furnace and insulating at a preset temperature, and measuring a size and depth change of the processed sample; and
determining a glass erosion resistance rate of the processed sample based on the size and depth change.

16. The method of claim 15, wherein a chemical component of the sample to be measured includes ZrO₂, SiO₂, and impurities, wherein a mass percentage of the ZrO₂ in the sample to be measured is not less than 93%, a mass percentage of the SiO₂ in the sample to be measured is not less than 4%, and a mass percentage of the impurities in the sample to be measured is greater than 0 and not greater than 3%, and a sum of the mass percentages of the ZrO₂, the SiO₂, and the impurities is 100%.

17. The method of claim 15, wherein the glass compound includes a glass raw material and a crushed glass.

18. The method of claim 17, wherein a mass fraction ratio of the glass raw material to the crushed glass is (75%-76%):(24%-25%).

19. The method of claim 15, wherein the preset temperature is 1600-1700° C.

20. The method of claim 15, wherein a duration of the insulation is 65-80 h.
